(19) United States
(12) Patent Application Publication
(10) Pub. No.: US 2019/0015302 A1
(43) Pub. Date: Jan. 17, 2019

(54) FLUORESCENT PARTICULATE MATERIAL

(71) Applicant: L'OREAL, Paris (FR)

(72) Inventors: Laurent VIDAL, Paris (FR); Julie MARTIN-BESNARDIERE, Aulnay-sous-Bois (FR)

(73) Assignee: L'OREAL, Paris (FR)

(21) Appl. No.: 16/066,898

(22) PCT Filed: Dec. 28, 2016
(86) PCT No.: PCT/EP2016/082795

§ 371 Date: Jun. 28, 2018

(30) Foreign Application Priority Data
Dec. 29, 2015 (FR) 1563417

(51) Int. Cl. A61K 8/02; C09K 11/06; C09C 1/30; A61K 8/25; A61K 8/58; A61Q 19/02; A61Q 19/08

(52) U.S. Cl. CPC A61K 8/025; C09K 11/06; C09C 1/3081; A61K 8/0279; A61K 8/25; A61K 8/585; A61K 2800/434; A61Q 19/08; C01P 2004/32; C01P 2004/61; A61K 2800/412; A61K 2800/612; A61Q 19/02

(57) ABSTRACT

The present invention relates to a fluorescent particulate material, formed from particles of an oxide of an element M, some of the atoms M of which bear a cationic group of formula (I) all or some of said cationic radicals of formula (I) being combined ionically with an anionic form of an organic compound A, which is fluorescent in the visible region, said anionic form of an organic compound A bearing at least one sulfonate group and/or at least one carboxylate group.

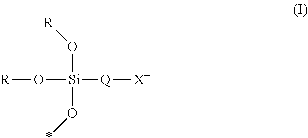

The present invention relates to cosmetic materials intended to be applied to keratin materials, in particular the skin, especially of the body, the hands, the neck, the face and the lips.

More precisely, these materials are transparent materials, which scatter light by transmission, and emit light by luminescence.

People whose skin has surface imperfections and irregularities such as colored marks, wrinkles, fine lines or dilated pores, often wish to hide these defects and, to do so, use cosmetic compositions containing light scatterers in the form of microparticles, for reducing the visibility of the skin imperfections.

This effect is known as the soft-focus effect. The criteria to be respected in order to obtain such a soft-focus effect are described by Emmert, Quantification of the Soft Focus Effect, Cosmetics and Toiletries, 111, 57-61, 1996. The particles must have minimum absorption in the visible range, high total transmission so as not to create a covering effect on the skin, this transmission being scattered so that the back-scattering produced by reflection of the rays by the skin appears as homogeneous as possible, minimum specular reflection, and high scattering reflection so as to have homogeneous light distribution.

In this respect, cosmetic compositions contain, as is usual, either boron nitride particles, or nylon particles, or polymer-covered aluminum platelets, or spherical silica particles. This being the case, these compositions do not make it possible to create really efficient and sufficiently perceptible masking of the relief irregularities. Specifically, fine lines are only slightly masked, whereas more pronounced wrinkles, such as expression wrinkles, are not masked. Furthermore, they give an unattractive matt appearance and do not produce a natural look perception.

Moreover, people with colored or even dark skin, who wish to lighten the color of their skin, and people who wish to attenuate or even eliminate skin dyschromia appearing either with age or after exposure to UV radiation, a pregnancy mask or another cutaneous pathology, use for this purpose cosmetic or dermatological compositions containing bleaching agents (such as hydroquinone and derivatives thereof). However, the effects of such bleaching agents appear only slowly, after they have been used repeatedly and in a prolonged manner. Moreover, since they act only on endogenous melanin biosynthesis, these bleaching agents do not make it possible to modify the shade of the lightening.

Cosmetic compositions which unify the skin complexion by giving it an immediate white appearance also exist. These compositions contain scattering white pigments which give them the opacity and covering necessary to obtain the desired effect. However, this covering power creates opacity which detracts from the natural appearance, transparency and lightness of the skin thus made up. It especially gives rise to a dull, grayish effect.

Cosmetic compositions comprising optical brighteners encapsulated in porous mineral particles are also known (FR 2 857 254, L'Oréal). However, the lightening effect on the skin color is insufficient, and the shade of this lightening does not vary or varies too little to be able to modify the shade of the lightening as a function of the various flesh tones.

There is thus a real need for cosmetic compositions that can afford immediate masking of skin relief irregularities, on wrinkles, especially fine lines and more visible wrinkles, such as expression wrinkles, while at the same time conserving a very natural appearance of the skin without any covering marks.

In particular, there is a real need for cosmetic materials that are capable of giving a pronounced soft-focus appearance and that can immediately reduce the visibility of fine lines and of more pronounced wrinkles, while at the same time ensuring a natural appearance.

There is also a real need for cosmetic materials that scatter light, mainly by transmission, and limit the back-scattering of cosmetic films containing them created on the skin.

Surprisingly, the inventors have observed that it is possible to obtain efficient and perceptible masking of skin relief irregularities immediately, especially on fine lines and more visible wrinkles, such as expression wrinkles, while at the same time maintaining a very natural look of the skin, without any covering marks, by using a specific particulate material incorporating a fluorophoric agent.

Thus, according to a first aspect, the present invention relates to a fluorescent particulate material, formed from particles of an oxide of an element M, some of the atoms M of which bear a cationic group of formula (I):

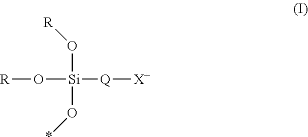

in which:
   “*-” representing a covalent bond with an atom M,
   M being chosen from titanium, iron, copper, zinc, zirconium, strontium, silicon, bismuth, cerium and mixtures thereof,
   Q represents a linear or branched divalent C₁-C₁₂ alkylene radical, optionally interrupted with one or more oxygen atoms, and optionally substituted, or an optionally substituted C₆-C₁₀ arylene radical, or a divalent radical —R′—C₆H₄—R″, R′ and R″, which may be identical or different, represent a divalent C₁-C₄ alkylene radical and —C₆H₄— represents an aromatic divalent radical,
   X⁺ represents a cationic organic group, and
   the groups R, which may be identical or different, represent a hydrogen atom, a C₁-C₆ alkyl radical or a covalent bond with an atom M,
all or some of said cationic radicals of formula (I) being combined ionically with an anionic form of an organic compound A, which is fluorescent in the visible region, said anionic form of an organic compound A bearing at least one sulfonate group and/or at least one carboxylate group.

As emerges from the examples below, the material of the invention makes it possible to obtain efficient and perceptible masking of skin relief irregularities immediately, especially on fine lines and more visible wrinkles, such as expression wrinkles, while at the same time maintaining a very natural look of the skin, without any covering marks.

As described below, some materials of the invention also make it possible to obtain immediate and modulable lightening of the skin complexion.

Consequently, according to a first aspect, the present invention relates to a cosmetic composition comprising, in a physiologically acceptable medium, at least one material in accordance with the invention.

A cosmetic composition comprising a material in accordance with the invention constitutes, for example, a product applied to the skin, especially the face, the neck and the hands, which imparts immediate color homogeneity thereto, by masking its colorimetric imperfections due to dyschromia marks or hypervascularized blood capillaries, and giving a natural look thereto.

According to another aspect, the present invention relates to a process for preparing a material in accordance with the invention, comprising at least the steps consisting in:
a) placing particles comprising at least one oxide of an element M in contact with at least one salt of formula (II):

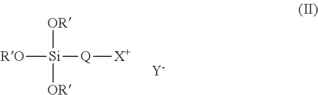

in which:
   M, Q and X⁺ are as defined above,
   R′ represents a linear or branched C₁-C₆ alkyl group, preferably methyl or ethyl group, and
   Y⁻ represents a monovalent anion, preferably chosen from halide
   anions, Y⁻ preferentially representing a chloride anion, under reaction conditions that are suitable for establishing covalent bonds between atoms M and cations of the salt of formula (II), and
b) placing in contact the grafted particles formed in step a) with at least one salt of an organic compound A as defined above, under reaction conditions that are suitable for ionic exchange between anions Y⁻ and anionic forms of said organic compound A.

Fluorescent Particulate Material

Material of the invention is characterized in that it is formed from particles of an oxide of an element M, some of the atoms M of which bear a cationic group of formula (I):

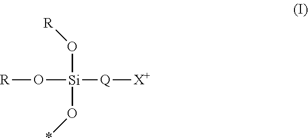

in which “*-”, M, Q, X⁺ and R are as defined in formula (I) above,

all or some of said cationic radicals of formula (I) being combined ionically with an anionic form of an organic compound A, which is fluorescent in the visible region, said anionic form of an organic compound A bearing at least one sulfonate group and/or at least one carboxylate group.

The ionic nature of the bond between the cationic groups and the anionic forms of the fluorescent organic compound A makes it possible, surprisingly, to increase the content of fluorescent organic compound A in the material according to the invention and thus to increase the fluorescence efficacy of said material.

Furthermore, the material of the invention has the advantage of having homogeneous distribution of the fluorescent organic compounds A on the particles. As a result, when it is applied to keratin materials, for instance the skin, the material in accordance with the invention allows visible light (400 nm-700 nm) to be transmitted by high scattering to give a natural look. The light becomes back-scattered very homogeneously by the keratin materials and with high intensity to give perceived homogeneity of the surface.

Furthermore, the material generates light via a luminescence phenomenon, so as overall to render perceptible the masking of the marked irregularities of the keratin materials, such as expression wrinkles.

The material is also transparent, which has the advantage of giving the skin a natural light, non-opaque and non-covering look.

Material of the invention is in the form of particles, which may be of spherical, cylindrical, ovoid, multifaceted polyhedral, platelet or needle shape, or in the form of aggregates.

The shape of the particles may be determined by electron microscopy observations.

The particles may have a mean size ranging from 300 nm to 100 μm and preferably from 500 nm to 20 μm.

The mean size of the particles may be determined by electron microscopy measurements.

The oxide of the element M may be chosen from the group formed by TiO₂, Fe₂O₃, CuO, ZnO, ZrO₂, SiO₂, Bi₂O₃, CeO₂, SrO₂ and SiₓO_{y}, x and y being independently comprised between 0.1 and 2.

Preferably, the oxide of the element M is a silicon oxide.

According to a particular embodiment, the particles are porous and are preferably porous silica particles.

The term “porous” means that the surface of the particles has pores that are accessible to the gases and liquids.

When they are porous, the particles have a porosity of less than 1000 m²/g and preferably greater than 10 m²/g.

The porosity of the particles may be determined via methods that are known by those skilled in the art, such as using a porosimeter.

Advantageously, the particles are porous silica particles having a mean pores size greater than 10 nm, preferably greater than 15 nm, more preferably greater than 20 nm. In particular, the mean pores size is lower than 50 nm, preferably lower than 40 nm, more preferably lower than 30 nm. Preferably, the mean pores size is ranging from 20 nm to 30 nm.

The mean pores size may be determined using a porosimeter via methods that are known by those skilled in the art. For example, a measurement method is described in the application EP-A-0407262. It has been noticed that materials according to the invention, which particles are porous silica particles having a mean pores size greater than 10 nm, also make it possible to obtain immediate and modulable lightening of the skin complexion, as emerges from the examples below.

As examples of porous silica, are those sold under the tradename SUNSPHERE H33 (porosity of 22.6 nm) and SUNSPHERE H53 (porosity of 22.4 nm) by the company AGC SI-TECH.

Preferably, at least one of the two groups R represents a bond with an atom M.

Thus, according to a particular embodiment, all or some of the cationic groups of formula (I) correspond to formula (I′):

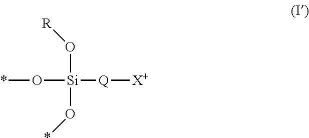

in which Q, X⁺ and R are as defined in formula (I).

The cationic group X⁺ of the cationic group of formula (I) may especially correspond to one of the two distinct variants detailed below.

According to a first variant, the group X⁺ has the formula —N⁺R₁R₂R₃ in which R₁, R₂ and R₃, which may be identical or different, independently represent a linear or branched C₁-C₆ alkyl radical, optionally interrupted with one or more oxygen atoms, and optionally substituted, or an optionally substituted C₆-C₁₀ aryl group, one of the groups R₁, R₂ and R₃ eventually forming a linear or branched C₁-C₂₀ alkyl radical, two of the groups R₁, R₂ and R₃ possibly forming together, with the nitrogen atom to which they are attached, an optionally substituted, 5- or 6-membered, saturated or unsaturated heterocycle.

Preferably, according to this variant, the groups R₁, R₂ and R₃, which may be identical or different, independently represent a methyl or ethyl group.

According to this variant, when two of the groups R₁, R₂ and R₃ together form, with the nitrogen atom to which they are attached, an optionally substituted 5- or 6-membered saturated or unsaturated heterocycle, said heterocycle is preferably saturated and represents, for example, a pyrrolidinium, piperidinium, morpholinium or piperazinium group.

When they denote a linear or branched C₁-C₆ alkyl radical, optionally interrupted with one or more oxygen atoms, the groups R₁, R₂ and R₃, which may be identical or different, may be substituted with one or more radicals chosen from the group consisting of the radicals: hydroxyl, C₁-C₂ alkoxy, amino of formula NR⁵R⁶, carboxyl, carboxylate of formula COOM¹ in which M¹ represents a lithium, sodium or potassium cation, —COOalkyl in which alkyl is C₁-C₄, carboxamido of formula CONR⁵R⁶, and sulfonamido of formula SO₂NR⁵R⁶.

R⁵ and R⁶, which may be identical or different, independently represent a radical chosen from the group consisting of a hydrogen atom; a linear or branched C₁-C₄ alkyl, optionally substituted with one or more radicals chosen from the group consisting of the radicals: hydroxyl, C₁-C₂ alkoxy, a carboxamido of formula CONR⁷R⁸, and a sulfonyl of formula SO₂R⁷; a C₆-C₁₀ aryl, optionally substituted with a group R⁹ as defined below; and a saturated or unsaturated 5- to 6-membered heterocycle, for instance an optionally substituted piperidyl, dihydropiperidyl or pyrrolidinyl ring.

R⁷ and R⁸, which may be identical or different, independently represent a radical chosen from the group consisting of a hydrogen atom, and a linear or branched C₁-C₄ alkyl radical, optionally interrupted with one or more hydroxyl or C₁-C₂ alkoxy radicals.

When they denote a C₆-C₁₀ aryl group, the groups R₁, R₂ and R₃, which may be identical or different, may be substituted with one or more radicals R⁹.

R⁹ represents a radical chosen from the group consisting of a hydroxyl radical; a C₁-C₂ alkoxy; a halogen; —COOalkyl in which alkyl is C₁-C₄; a carboxamido of formula CONR⁵R⁶; a sulfonamido of formula SO₂NR⁵R⁶; and a linear or branched C₁-C₄ alkyl, optionally interrupted with one or more oxygen atoms, and optionally substituted with one or more radicals chosen from the group consisting of the radicals: hydroxyl, C₁-C₂ alkoxy, amino of formula NR⁵R⁶, carboxyl, —COOalkyl in which alkyl is C₁-C₄, carboxamido of formula CONR⁵R⁶, and sulfonamido of formula SO₂NR⁵R⁶.

R⁵ and R⁶, which may be identical or different, are as defined above.

When two of the groups R₁, R₂ and R₃ together form, with the nitrogen atom to which they are attached, a 5- or 6-membered heterocycle, said heterocycle may be substituted with one or more groups R⁴ as defined below.

According to another variant, the group X⁺ represents an unsaturated, preferably aromatic, 5- or 6-membered heterocyclic group, comprising one or more heteroatoms including a nitrogen atom bearing a positive charge, said group being optionally substituted.

Preferably, according to this variant, the group X⁺ is chosen from the group consisting of imidazolium, oxazolium, thiazolium, pyridinium, pyrimidinium, pyrazinium, benzimidazolium, benzoxazolium and benzothiazolium groups.

Preferentially, according to this variant, the group X⁺ represents an imidazolium group.

When the group X⁺ represents an unsaturated, preferably aromatic, 5- or 6-membered heterocyclic group, comprising one or more heteroatoms including a nitrogen atom bearing a positive charge, said group may be substituted with one or more groups R⁴.

R⁴ represents a radical chosen from the group consisting of a linear or branched C₁-C₆ alkyl, optionally interrupted with one or more oxygen atoms, and optionally substituted with one or more radicals chosen from the group consisting of the radicals: hydroxyl, C₁-C₂ alkoxy, amino of formula NR⁵R⁶, carboxyl, —COOalkyl in which alkyl is C₁-C₄, carboxamido of formula CONR⁵R⁶, sulfonamido of formula SO₂NR⁵R⁶; a hydroxyl; a C₁-C₂ alkoxy; a carboxamido of formula CONR⁷R⁸; a sulfonyl of formula SO₂R⁷; an amino of formula NR⁵R⁶; and a C₁-C₆ aryl radical, optionally substituted with one or more radicals R⁹.

R⁵, R⁶, R⁷, R⁸ and R⁹ are as defined above.

When Q represents a linear or branched divalent C₁-C₁₂ alkylene radical, optionally interrupted with one or more oxygen atoms, said radical may be substituted with one or more radicals chosen from the group consisting of the radicals: hydroxyl, C₁-C₂ alkoxy, amino of formula NR⁵R⁶, —COOalkyl in which alkyl is C₁-C₄, carboxamido of formula CONR⁵R⁶, and sulfonamido of formula SO₂NR⁵R⁶.

R⁵ and R⁶ are as defined above.

When Q represents a C₆-C₁₀ arylene radical, said radical may be substituted with one or more radicals R⁹ as defined above.

Preferably, Q represents a linear or branched divalent C₁-C₆ alkylene radical.

According to the present invention, the “alkyl” radicals represent saturated straight or branched-chain hydrocarbon-based radicals, comprising from 1 to 6 carbon atoms, preferably from 1 to 4 carbon atoms (they may typically be represented by the formula CₙH₂ₙ₊₁, n representing the number of carbon atoms). When they are linear, mention may be made especially of methyl, ethyl, propyl, butyl, pentyl and hexyl radicals. When they are branched or substituted with one or more alkyl radicals, mention may be made especially of isopropyl and tert-butyl radicals.

The “alkoxy” radicals according to the present invention are radicals of formula —O— alkyl, the alkyl group being as defined previously.

The “carboxyl” radical denotes —COOH.

The “5- or 6-membered heterocyclic” radicals denote 5- to 6-membered cyclic systems, comprising one or more heteroatoms chosen from N, O and S, and from 1 to 4 or 5 carbon atoms, preferably 4 or 5. A heterocycle may be saturated or unsaturated. An unsaturated heterocycle may be partially unsaturated or aromatic.

The term “aryl” denotes a monocyclic or bicyclic hydrocarbon-based aromatic system, preferably of 6 to 10 carbon atoms. Among the aryl radicals, mention may be made especially of phenyl and naphthyl radicals.

The “halogens” especially denote F, Cl, Br and I atoms.

Organic Compound A

In the material of the invention, all or some of the cationic radicals of formula (I) is combined ionically with an anionic form of an organic compound A, which is fluorescent in the visible region, said anionic form of an organic compound A bearing at least one sulfonate group and/or at least one carboxylate group.

Said anionic forms are combined ionically with the cationic groups X⁺ as defined above.

The term “all or some” means that certain cationic groups X⁺ may be combined with other counter-anions to ensure the overall electronic neutrality of the material. They may be halide anions, such as chloride, fluoride, bromide or iodide anions, preferably chloride.

According to one embodiment, the anionic form of the organic compound A bears from 2 to 4 groups chosen from the group consisting of the sulfonate group (—SO₃⁻), the carboxylate group (—COO⁻), and mixtures thereof.

The organic compound A is preferably a fluorophoric compound chosen independently from the list in the European Cosmetic Directive; the FDA list; and the “Fluorescent Whitening Agent, Encyclopedia of Chemical Technology”, Kirk-Othmer, 4th.11:227-241, 1994.

The organic compound A belongs, for example, to a chemical family chosen from the group consisting of xanthenes (such as benzo[a]xanthenes, benzo[b]xanthenes, benzo[c]xanthenes), coumarins (such as benzocoumarins), phenoxazines (such as benzo[a]phenoxazines, benzo[b]phenoxazines, benzo[c]phenoxazines), phenothiazines (such as benzo[b]thiazines, benzo[c]thiazines), naphthalimides, naphtholactams, lactamimides, quinacridones, epindolines, thio-epindolines, phthalimides, oxazolones, benzotriazoles, diphenylmaleimides, dibenzofurans, pyrimidines, triazines, 1,3,5-triazin-2-yl derivatives, pyrazines, triazoles, methines, distyrylbenzenes, distyrylbiphenyls, divinylstilbenes, triazinylaminostilbenes, stilbenyl-2H-triazoles, benzoxazoles, benzofurans, benzimidazoles, 1,3-diphenyl-2-pyrazolines, diketopyrrolopyrroles, perylenes and perylenemonoimides.

The organic compound A may in particular belong to one of the following families:

a—The fluorophores of the xanthene family are represented by formula (F-I):

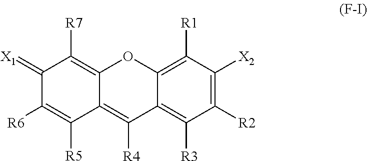

in which:

X₁ represents an oxygen atom, a radical N—Z₁ or N⁺Z₁Z₂M₁⁻,

X₂ represents a hydroxyl radical or a radical NZ₁Z₂,

Z₁ and Z₂, independently of each other, represent a radical chosen from the group Gp₁ consisting of a hydrogen atom; a linear or branched C₁-C₅ alkyl, optionally interrupted with one or more oxygen atoms, and optionally substituted with one or more radicals chosen from the group consisting of a radical OR₈, a radical NR₉R₁₀, a carboxyl radical, a radical COOM₁, a halogen, a sulfonic radical, a carboxamido radical CONR₉R₁₀ and a sulfonamido radical SO₂NR₉R₁₀,

Z₁ and Z₂ may form, with the nitrogen atom to which they are attached, a saturated or unsaturated 5- to 7-membered heterocycle optionally substituted with one or more radicals chosen from the group Gp₂ consisting of halogen atoms; the radicals: amino, (di)(C₁-C₄)alkylamino, hydroxyl, carboxyl, carboxamido and (C₁-C₂)alkoxy, C₁-C₄ alkyl radicals optionally substituted with one or more hydroxyl, amino, (di)alkylamino, alkoxy, carboxyl or sulfonyl radicals; the ring not comprising any peroxide bonds or any diazo or nitroso radicals,

X₁ (or, respectively, X₂) may be optionally linked to R₆ and/or R₇ (or, respectively, to R₁ or R₂), to form together a saturated or unsaturated 5- to 6-membered heterocycle, for instance an optionally cationic piperidyl, dehydropiperidyl or pyrrolidinyl ring in which the associated anion or mixture of anions is chosen from the group consisting of a halide such as chloride, bromide, fluoride or iodide, a hydroxide, a sulfate, a hydrogen sulfate, an alkyl sulfate for which the linear or branched alkyl part is C₁-C₆, such as the methyl sulfate or ethyl sulfate ion, a carbonate, a hydrogen carbonate; carboxylates such as formate, acetate, citrate, tartrate or oxalate, alkylsulfonates for which the linear or branched alkyl part is C₁-C₆, such as the methylsulfonate ion, arylsulfonates for which the aryl part, preferably phenyl, is optionally substituted with one or more C₁-C₄ alkyl radicals, for instance 4-tolylsulfonate,

R₁, R₂, R₃, R₅, R₆ and R₇, which may be identical or different, represent a radical chosen from the group Gp₃ consisting of a hydrogen atom; a halogen; a C₁-C₄ alkyl optionally substituted with one or more radicals chosen from the group consisting of a radical OR₈, a radical NR₉R₁₀, a carboxyl radical, a sulfonic radical, a carboxamido radical CONR₉R₁₀, a sulfonamido radical SO₂NR₉R₁₀; a carboxyl radical; a carboxamido radical (RCONH—); a (C₁-C₄)alkylsulfonyl radical (SO₂R); an alkylsulfonamido radical ((C₁-C₄)alkyl)SO₂NH—); a sulfonic radical (—SO₃H); a (C₁-C₄)alkyl sulfoxide radical (—SOR); an aminosulfonyl radical (NH₂—SO₂—); a hydroxyl radical; a C₁-C₄ alkoxy radical; a C₂-C₄ hydroxyalkoxy radical; a radical NR₁₃R₁₄; an aryl radical optionally substituted with one or more radicals chosen from the group Gp₄; a heteroaryl radical optionally substituted with one or more radicals chosen from the group Gp₄; a cationic aza-heteroaryl radical optionally substituted with one or more radicals chosen from the group Gp₄,

R₅ and R₆ may form a saturated or unsaturated 6-membered carbocycle or heterocycle optionally substituted with one or more radicals chosen from the group Gp₂,

R₄ represents a radical from among: hydrogen, halogen; trifluoromethyl; linear or branched C₁-C₈ alkyl; linear or branched C₂-C₈ alkenyl; aryl optionally substituted with one or more radicals chosen from the group Gp₄; heteroaryl optionally substituted with one or more radicals chosen from the group Gp₄; naphthyl optionally substituted with one or more radicals chosen from the group Gp₄,

R₈, R₉ and R₁₀, which may be identical or different, represent a hydrogen atom; a linear or branched C₁-C₄ alkyl radical optionally substituted with one or more radicals chosen from the group consisting of a hydroxyl, a C₁-C₂ alkoxy, a carboxamido CONR₁R₁₂, a sulfonyl SO₂R₁₁,

R₁₁ and R₁₂, which may be identical or different, represent a hydrogen atom; a linear or branched C₁-C₄ alkyl radical optionally substituted with one or more hydroxyl or C₁-C₂ alkoxy;

the group Gp₄ consists of a halogen; hydroxyl; C₁-C₄ alkoxy; carboxyl; carboxamido; (C₁-C₄)alkylsulfonyl (—SO₂-alkyl); sulfonic (—SO₃H); sulfonate SO₃M′ in which M′ represents a lithium, sodium or potassium ion, sulfate (—OSO₃H⁻); alkylsulfoxide (—SO-alkyl); alkylsulfonamido ((C₁-C₄)alkylSO₂NH—); dialkylamino NR₁₃R₁₄; nitro; optionally substituted phenyl; C₁-C₄ alkyl optionally substituted with one or more radicals chosen from the radicals: hydroxyl, C₁-C₄ alkoxy, carboxyl, carboxamido, (C₁-C₄)alkylsulfonyl, sulfonic, alkyl sulfoxide, alkylsulfonamido or NR₁₃R₁₄; and optionally the salts thereof, and the solvates thereof,

R₁₃ and R₁₄ represent, independently of each other, a radical chosen from the group Gp₁. They may form, with the nitrogen atom that bears them, a 5- to 7-membered ring, and

M₁ represents a lithium, sodium, potassium, calcium, magnesium or ammonium cation, mono-, di-, tri- or tetra-substituted with a C₁-C₈ alkyl or C₂-C₄ hydroxyalkyl.

The particular compounds of formula (F-I) may, in certain cases, be represented by the isomeric forms thereof according to formula (F-I′), in which n represents an integer from 0 to 4.

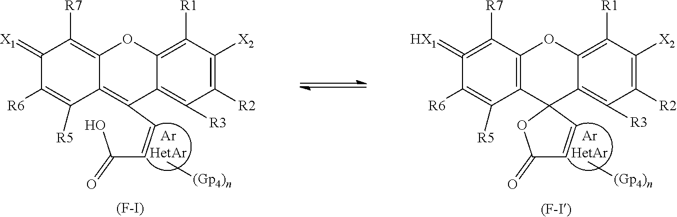

In formulae (F-I) and (F-I′), at least one of the radicals R₁, R₂, R₃, R₅, R₆ or R₇ represents a sulfonic acid radical SO₃H.

The preferred compounds of formula (F-I) are the following (or the acid form thereof):

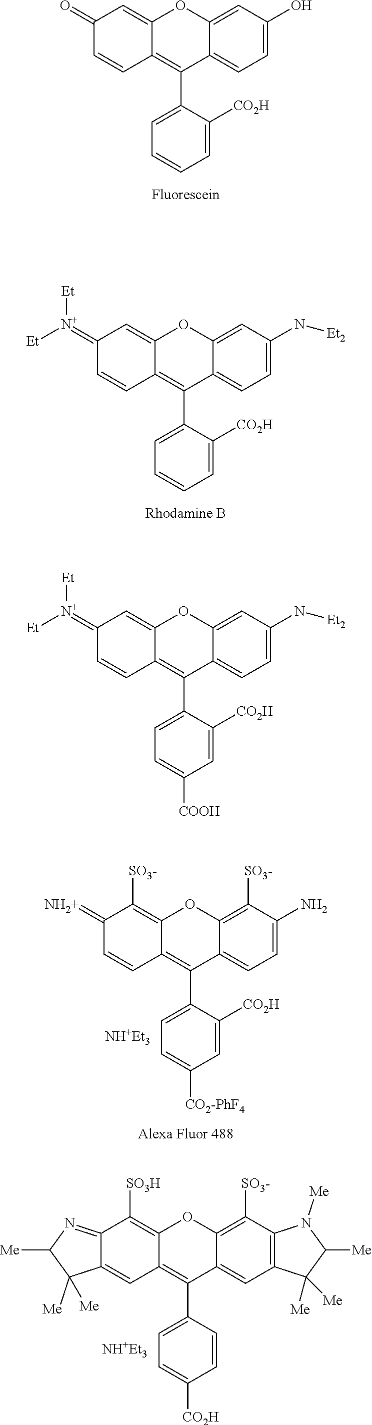

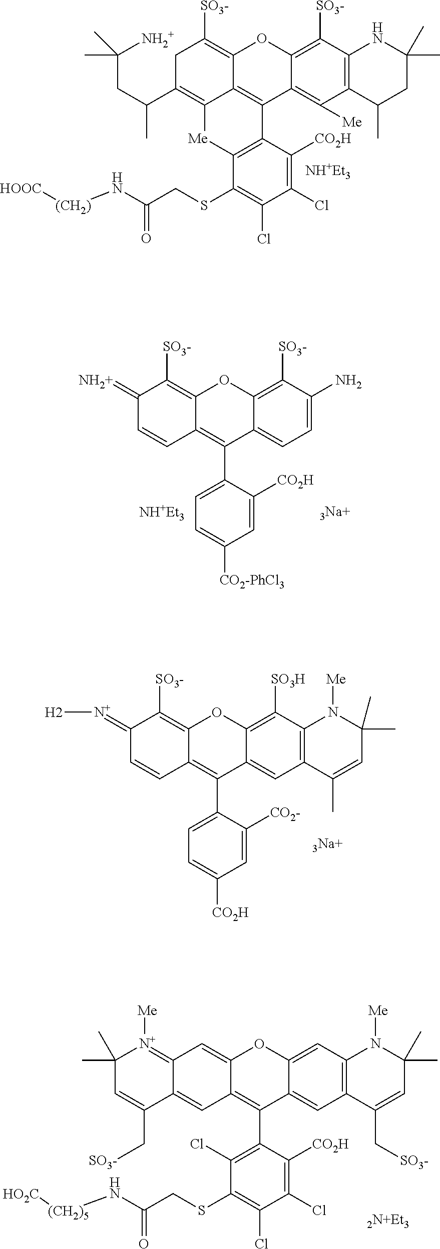

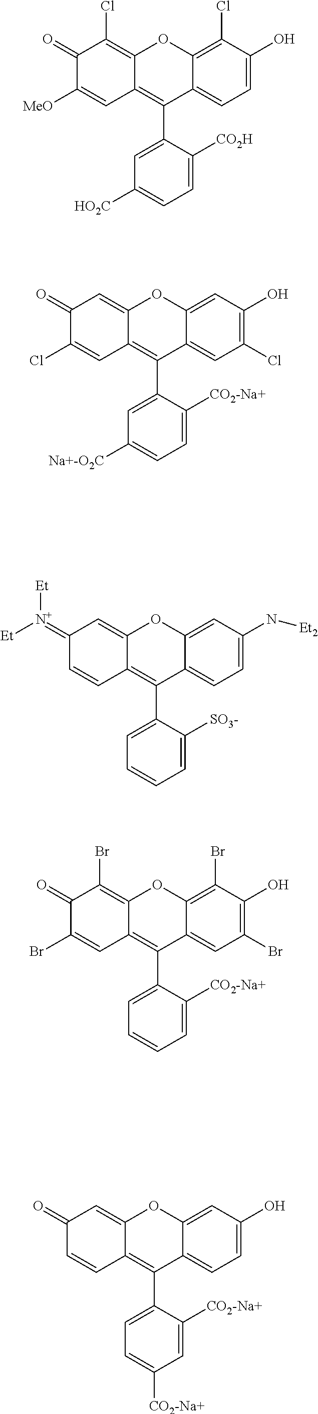

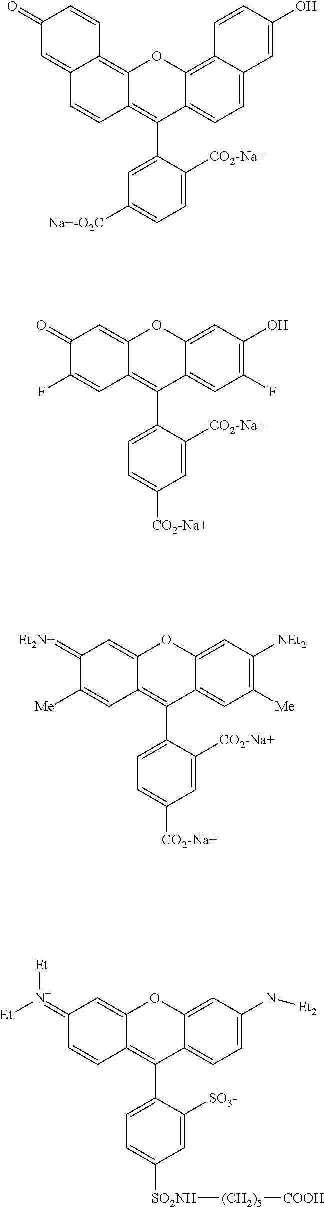

b—The fluorophores of the benzo[b]xanthene and benzo[c]xanthene families are represented, respectively, by formulae (F-IIa) and (F-IIb):

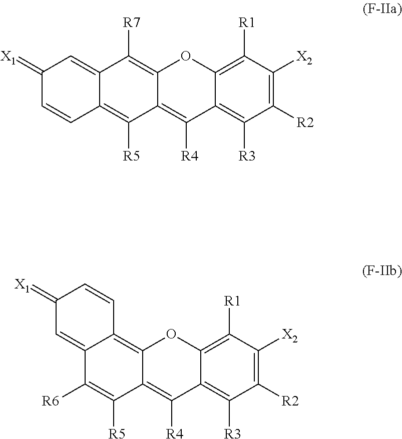

and the isomeric forms thereof according to a representation similar to that of (F-I) ⇄(F-I′),

in which R₁, R₂, R₃, R₄, R₅, R₆, R₇, X₁ and X₂ are as defined in (F-I).

For example, these isomeric forms are represented by the following formulae:

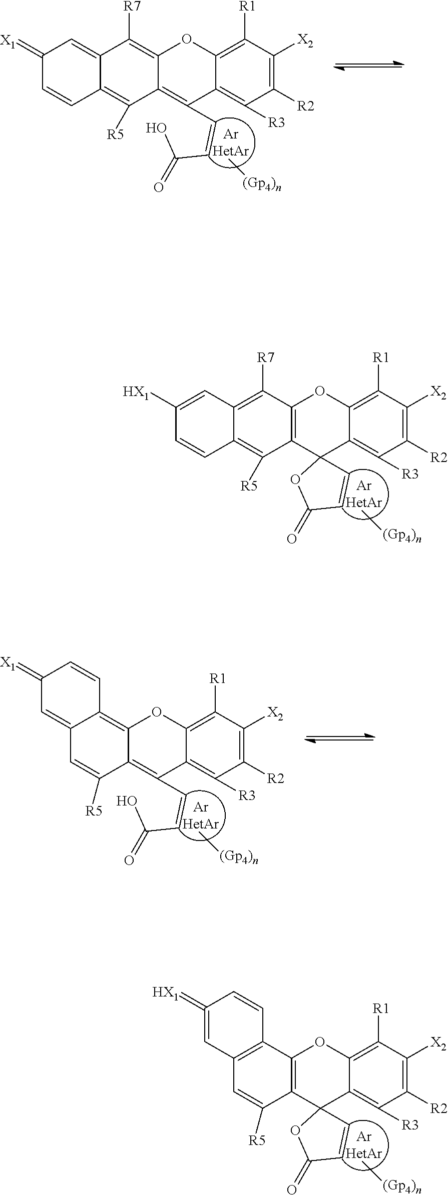

in which at least one of the radicals R₁, R₂, R₃, R₅, R₆ or R₇ represents a sulfonic acid SO₃H or carboxylic acid CO₂H radical.

c—The fluorophores of the coumarin family are represented, respectively, by formulae (F-III):

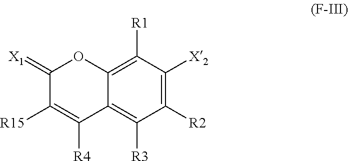

in which R₁₅ is defined by the same radicals as R₁ in (F-I),

X′₂ represents a hydroxyl radical, a C₁-C₂ alkoxy radical; or a radical NZ₁Z₂ with the proviso that Z₁ and Z₂ do not together represent a hydrogen atom,

X′₂ and R₂ together may optionally form an optionally unsaturated 5- or 6-membered, ring, optionally substituted with a radical of the group Gp₃,

at least one of the radicals R₁, R₂, R₃ or R₁₅ representing or containing a sulfonic acid SO₃H or carboxylic acid CO₂H radical.

The preferred compounds of formula (F-III) are the following (or the acid form thereof):

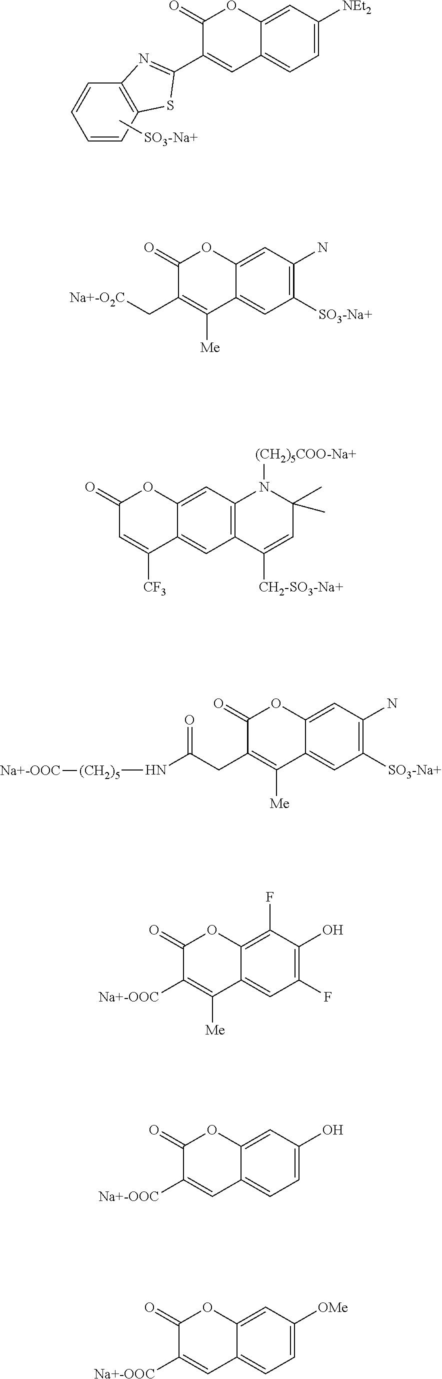

d—The fluorophores of the naphthalimide family are represented by formula (F-x):

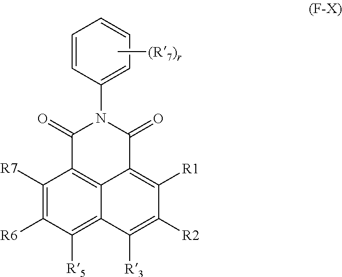

in which:

R′₇ represents a radical chosen from the group Gp₄;

R′₃ and R′₅ represent radicals as defined by R₃ and R₅, and may optionally form a lactam ring, the nitrogen atom of which is optionally substituted with a group Z₁;

r represents an integer between 0 and 5,

at least one of the radicals R₁, R₂, R′₃, R′₄, R₆, R₇ or R′₇ representing or containing a sulfonic acid SO₃H or carboxylic acid CO₂H radical.

The preferred compounds of formula (F-X) are the following (or the acid form thereof):

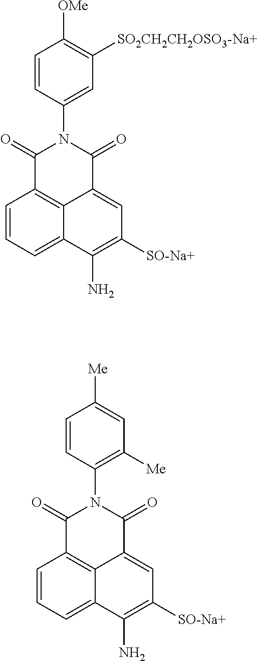

e—The fluorophores of the naphtholactam family are represented by formula (F-XI):

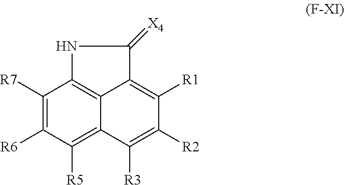

in which X₄ represents a radical as defined by XI; a heterocyclic radical; a C₁-C₄ alkyl radical optionally substituted with one or more radicals chosen from the radicals: hydroxyl, C₁-C₄ alkoxy, cyano; carboxyl, carboxamido, (C₁-C₄)alkylsulfonyl, sulfonic, alkyl sulfoxide, alkylsulfonamide; aryl optionally substituted with one or more radicals chosen from the group Gp₄; a heteroaryl radical optionally substituted with one or more radicals chosen from the group Gp₄;

at least one of the radicals R₁, R₂, R₃, R₅, R₆ or R₇ representing or containing a sulfonic acid SO₃H or carboxylic acid CO₂H radical.

The preferred compounds of formula (F-XI) are the following (or the acid form thereof):

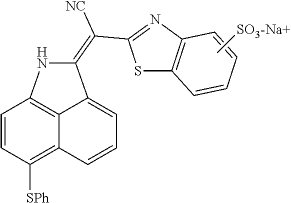

f—The fluorophores of the lactamimide family are represented by formula (F-XII):

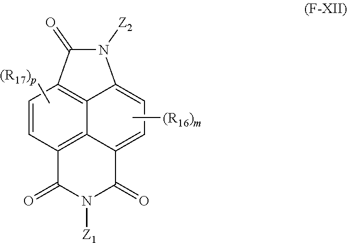

in which R₁₆ and R₁₇ represent radicals as defined by the radical R₁;

m and p represent an integer between 0 and 2,

at least one of the radicals R₁₆ or R₁₇ representing or containing a sulfonic acid SO₃H or carboxylic acid CO₂H radical.

g—The fluorophores of the quinacridone, epindoline and thio-epindoline families are represented, respectively, by formulae (F-XIII), (F-XIV) and (F-XV)

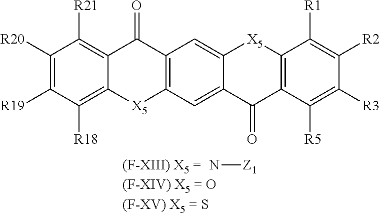

in which X₅ represents an oxygen or sulfur atom or a group N-Z₁;

R₁₈, R₁₉, R₂₀ and R₂₁ represent radicals as defined by the radical R₁, at least one of the radicals R₁, R₂, R₃, R₅, R₁₈, R₁₉, R₂₀ or R₂₁ representing or containing a sulfonic acid SO₃H or carboxylic acid CO₂H radical.

The quinacridone-based compounds that can emit white are described in WO 04/039 805.

h—The fluorophores of the phthalimide family are preferably represented by formula (F-XV):

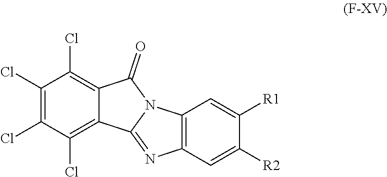

at least one of the radicals R₁ or R₂ representing or containing a sulfonic acid SO₃H or carboxylic acid CO₂H radical.

i—The fluorophores of the oxazolone family are preferably represented by formula (F-XVI):

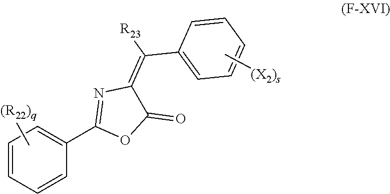

where:

R₂₂ and R₂₃ represent, independently of each other, a radical chosen from the group Gp₄,

s represents an integer between 0 and 5,

at least one of the radicals R₂₂ or X₂ representing or containing a sulfonic acid SO₃H or carboxylic acid CO₂H radical.

j—The fluorophores of the pyrimidine, triazine, triazole, pyrazine and dibenzofuran families are described in the publications WO 04/039 786, EP 2004/050 146, WO 05/023 960, EP 2004/052 984, EP 2005/051 731, EP 05/103 497 and EP 05 104 599.

k—The fluorophores of the stilbene family are represented by formula (F-XVII):

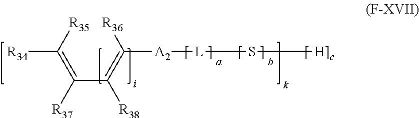

in which:

i represents an integer equal to 0 or 1;

k represents an integer equal to 1 or 2;

a, b and c represent an integer equal to 0 or 1,

and such that (a,b,c,k)=(0,0,1,1); (0,0,0,2), (1,0,0,1), (1,0,0,2), (1,1,0,2) or (1,1,1,1),

R₃₄ and A₂ represent an aryl radical optionally substituted with one or more radicals chosen from the group Gp₅; an aromatic heterocyclic radical optionally substituted with one or more radicals chosen from the group Gp₅; a cationic aromatic heterocyclic radical optionally substituted with one or more radicals chosen from the group Gp₅,

the group Gp₅ consists of a hydrogen atom; a linear or branched C₁-C₄ alkyl optionally substituted with one or more radicals chosen from the group consisting of a radical OR₈, SR₈, NR₉R₁₀, carboxyl, sulfonic, carboxamido CONR₉R₁₀, sulfonamido SO₂NR₉R₁₀; a —CH═CH—CN radical; a radical —CH═CH—CO₂R₈; a radical —CH═CH—Ar in which Ar represents an aryl group optionally substituted with a nitrile; a carboxyl radical; a (C₁-C₃)alkoxycarbonyl radical; a cyano radical; a halogen radical; a carboxamido radical (RCONH—); a (C₁-C₄)alkylsulfonyl radical (SO₂R); an alkylsulfonamido radical ((C₁-C₄)alkylSO₂NH—); a sulfonic radical (—SO₃H); a sulfonate radical (—SO₃M, in which M represents a sodium, potassium or lithium ion); a C₁-C₄ thioether radical; a (C₁-C₄)alkylsulfoxide radical (—SOR); an aminosulfonyl radical (NH₂—SO₂—); a hydroxyl radical; a C₁-C₄ alkoxy radical; a C₂-C₄ hydroxyalkoxy radical; a radical NR₃₉R₄₀; an aryl radical optionally substituted with one or more radicals chosen from the group Gp₄; a heteroaryl radical such as 1,2,3-triazole, 1,2,4-triazole, benzotriazole, benzoxazole, pyrazole, pyrazoline, pyrazolidine, indazole, imidazole, pyrrole, indole, pyrrolidine, indoline or oxadiazole, said radical being optionally substituted with one or more radicals chosen from the group Gp₄,

R₃₉ and R₄₀ represent, independently of each other, a radical chosen from the group Gp₁ and in particular a triazinyl group optionally substituted with a radical chosen from the group Gp₆ consisting of a C₁-C₄ alkoxy radical; NR₄₁R₄₂; C₁-C₄ alkyl optionally substituted with one or more radicals chosen from the radicals: hydroxyl, C₁-C₄ alkoxy, carboxyl, carboxamido, (C₁-C₄)alkylsulfonyl, sulfonic, alkylsulfoxide, alkylsulfonamido or NR₄₃R₄₄,

R₃₉ and R₄₀ may together form a 5- to 7-membered carbocycle or heterocycle with the nitrogen atom that bears them,

R₄₁, R₄₂, R₄₃ and R₄₄ represent, independently of each other, a radical chosen from the group Gp₁,

R₄₁ and R₄₂, on the one hand, and R₄₃ and R₄₄, on the other hand, may together form a 5- to 7-membered carbocycle or heterocycle with the nitrogen atom that bears them, such as, without being limited thereto, a pyrrolidine, morpholine or thiomorpholine ring,

R₃₅, R₃₆, R₃₇ and R₃₈ represent, independently of each other, a hydrogen radical; a C₁-C₈ alkyl optionally substituted with one or more radicals chosen from the group consisting of a radical OR₈, a carboxyl radical, a sulfonic radical and in which the carbon-based chain may optionally be interrupted with one or more oxygen atoms; carboxyl; cyano,

R₃₅ and R₃₆ (in the case where i=1) or R₃₅ and R₃₇ (in the case where i=0) may be linked together to form a 5-membered heterocycle chosen from furan, thiophene and pyrrole; a 6- or 10-membered carbocycle chosen from aryl and naphthyl,

R₃₄ and R₃₇ may be linked together to form a heterocycle chosen from benzofuran, benzothiophene, indole and azaindole,

L represents a linear or branched C₂-C₁₀ alkyl radical, the carbon-based chain of which may optionally be interrupted with at least one oxygen atom, optionally substituted with a C₂-C₈ alkoxy radical; a (di)(C₂-C₈)alkylamino radical, and

R₃₄ and A₂ preferably represent, independently of each other, a phenyl, naphthyl, pyridine, pyrimidine, imidazole, pyrazole, pyrrole, triazole, benzoxazole, benzimidazole, indole, azaindole, oxazolium, thiazolium, pyridinium, pyrimidinium, imidazolium, benzimidazolium, pyrazolium, pyrrolium, triazolium, oxazolium or thiazolium radical.

Preferably, mention may be made of styrylstilbene, triazinostilbene, hydroxycoumarin, aminocoumarin, oxazole, benzoxazole, imidazole, triazole, pyrazoline, pyrene and porphyrin derivatives.

The following compounds preferentially, but in a non-limiting manner, illustrate this family:

| | 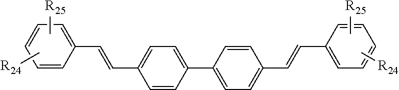 | | |
|---|---|---|---|
| | (R₂₄, R₂₅) = (SO₃Na(3), Cl(4)); (OMe (2), H); (SO₃Na | | |
| | (2), H). | | |
| 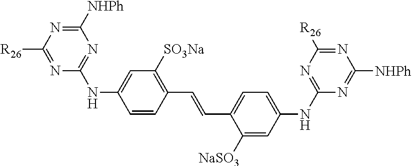 | 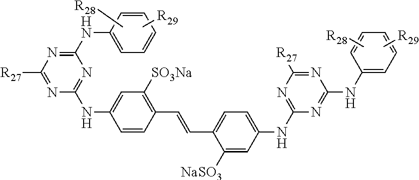 | | |
| | | R₂₈ | R₂₉ |
| | R₂₇ | (position) | (position) |
| R₂₆ = OMe, NHMe, NHEt, NH—CH₂—CH₂OH, | NH—CH₂—CH₂OH | SO₃Na (3) | H |
| NH(CH₃)(CH₂—CH₂—OH), N-morpholinyl, | N—(CH₂—CH₂—OH)₂ | SO₃Na (3) | H |
| NHPh, | N—(CH₂—CH₂OH)₂ | SO₃Na (4) | H |
| | N—(CH₂—CH₂OH)₂ | SO₃Na (2) | SO₃Na (5) |
| | N—(CH₂CH(OH)CH₃)₂ | SO₃Na (4) | H |
| | N(CH₃)(CH₂CH₂OH) | SO₃Na (4) | H |
| | NEt₂ | SO₃Na (2) | SO₃Na (5) |
| | N-morpholinyl | SO₃Na (2) | SO₃Na (5) |
| | N(CH₂CO₂Na)₂ | SO₃Na (4) | H |

| | |
|---|---|
| 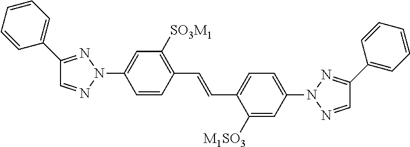 | 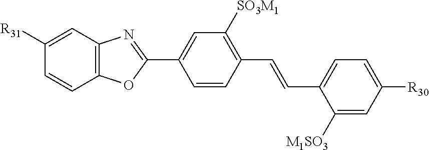 |
| M₁ = Na, K | (R₃₀, R₃₁) = (Me, COOMe) |
| | (H, methyloxadiazole) |

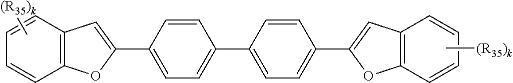

R₃₅ = SO₃Na
k = 1, 2

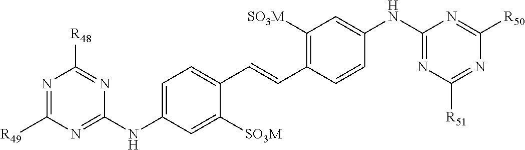

| R₄₈ | R₄₉ | R₅₀ | R₅₁ | M |
|---|---|---|---|---|
| Amino | —N—(CH₂CH₂OH)₂ | Amino | —N—(CH₂CH₂OH)₂ | Na |
| Amino | —N—Et(CH₂CH₂OH) | Amino | —N—Et(CH₂CH₂OH) | Na |
| Amino | —NH—CH₂—COONa | Amino | —NH—CH₂—COONa | Na |
| Amino | —N—Me(CH₂CH₂OH) | Amino | —N—Me(CH₂CH₂OH) | Na |
| Amino | —N-morpholino | Amino | —N-morpholino | Na |
| Amino | —NMe—CH₂—COONa | Amino | —NMe—CH₂—COONa | Na |
| Amino | —NH—(CH₂CH₂OMe) | Amino | —NH—(CH₂CH₂OMe) | Na |
| —N—(CH₂CH₂OH)₂ | —N—(CH₂CH₂OH)₂ | —N—(CH₂CH₂OH)₂ | —N—(CH₂CH₂OH)₂ | Na |
| —NH—(CH₂CH₂OH) | —NH—(CH₂CH₂OH) | —NH—(CH₂CH₂OH) | —NH—(CH₂CH₂OH) | Na |
| —NH—(CH₂CH₂OH) | —N-morpholino | —NH—(CH₂CH₂OH) | —N-morpholino | NH4⁺ |
| Amino | —NH—(CH₂CH₂CO₂Na) | Amino | —NH—(CH₂CH₂COONa) | Na |
| Amino | —NH—(CH₂CH₂OH) | Amino | —NH—(CH₂CH₂OH) | Na |
| Amino | —N-morpholino | Amino | —NH—(CH₂CH₂OMe) | Na |
| Amino | —N-morpholino | Amino | —NH—(CH₂CH₂OH) | Na |
| Amino | —N-morpholino | Amino | —NH—CH₂—COOH | Na |

The following fluorophores are also preferred:

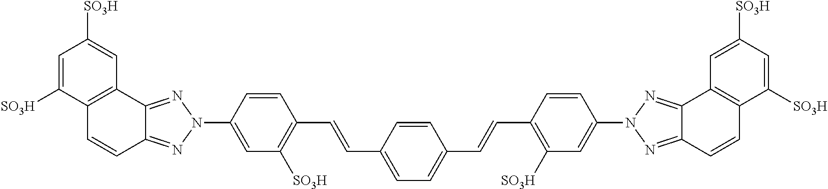 [SMILES: O=S(=O)(O)c1cc(S(=O)(=O)O)c2ccc3nn(-c4ccc(/C=C/c5ccc(/C=C/c6ccc(-n7nc8ccc9c(S(=O)(=O)O)cc(S(=O)(=O)O)cc9c8n7)cc6S(=O)(=O)O)cc5)c(S(=O)(=O)O)c4)nc3c2c1]

C.I. Direct Yellow 96

| 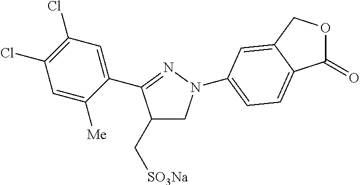 | 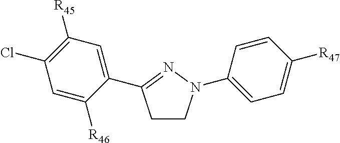 | | |
|---|---|---|---|
| | R₄₅ | R₄₆ | R₄₇ |
| | H | H | SO₂CH₂CH₂SO₃Na |
| | H | H | CO₂Na |
| | Cl | Me | SO₂CH₂CH₂SO₃Na |

Preferably, the organic compound A belongs to a chemical family chosen from the group consisting of stilbenes, xanthenes, coumarins and naphthalimides.

Preferentially, the organic compound A belongs to a chemical family chosen from the group consisting of stilbenes and xanthenes, advantageously stilbenes.

According to a particular variant of the invention, the organic compound A corresponds to the following formula:

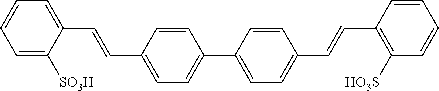 [SMILES: O=S(=O)(O)c1ccccc1/C=C/c1ccc(-c2ccc(/C=C/c3ccccc3S(=O)(=O)O)cc2)cc1]

The organic compound A is, for example, Tinopal® CBS-X (disodium 2-[(Z)-2-[4-[4-[(Z)-2-(2-sulfonatophenyl)ethenyl]phenyl] phenyl]ethenyl]benzenesulfonate) sold by BASF.

According to another particular variant of the invention, the organic compound A corresponds to the following formula:

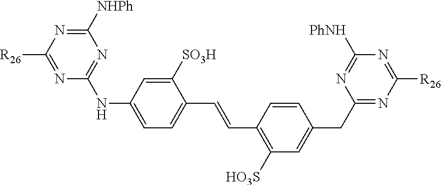

in which R₂₆ is a group chosen from OMe, NHMe, NHEt, NH—CH₂—CH₂OH, NH(CH₃)(CH₂—CH₂—OH), N-morpholinyl and NHPh.

R₂₆ preferably represents an N-morpholinyl group.

Process for Preparing the Material of the Invention According to another of its aspects, the present invention also relates to a process for preparing a material in accordance with the invention, comprising at least the steps consisting in:
a) placing particles comprising at least one oxide of an element M in contact with at least one salt of formula (II):

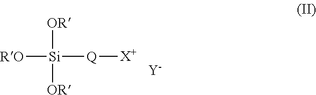

in which:
   M, Q and X⁺ are as defined above,
   R′ represents a linear or branched C₁-C₆ alkyl group, preferably methyl or ethyl group, and
   Y⁻ represents a monovalent anion, preferably chosen from halide anions, Y⁻ preferentially representing a chloride anion, under reaction conditions that are suitable for establishing covalent bonds between atoms M and cations of the salt of formula (II), and
b) placing in contact the grafted particles formed in step a) with at least one salt of an organic compound A as defined above, under reaction conditions that are suitable for ionic exchange between anions Y⁻ and anionic forms of said organic compound A.

The anionic organic part of the salt used bears at least one sulfonate group and/or at least one carboxylate group.

Preferably, the salt of the organic compound A is an alkali metal salt, preferably the sodium salt.

Step a) of the preparation process of the invention consists of a step of grafting, on to the atoms M at the surface of the particles, groups of formula:

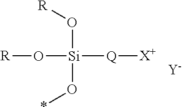

in which Q, X⁺ and Y⁻ are as defined in formula (II) and R is as defined in formula (I).

During this step, the units Si—OR′ are either converted into Si—OH units (in the case where R═H), or converted to form a bond with an atom M (formation of a unit Si—O-M), or are not modified (in the case where R═C₁-C₆ alkyl).

In general, during step a), at least two units Si—OR′ are converted to form two bonds with two atoms M (formation of two units Si—O-M).

A mixture of particles and of salt of formula (II) is generally prepared in an organic solvent, which is preferably apolar, for instance heptane.

The mixture is preferably heated, for example to the boiling point of the solvent, for a time necessary for disappearance of the salt of formula (II).

On conclusion of the reaction, the mixture is typically filtered and the recovered solid is washed with an organic solvent such as ethanol, and then dried.

Step b) of the process of the invention consists of an ionic exchange between the anions Y⁻ and the anionic forms of the organic compound A bearing at least one sulfonate group and/or at least one carboxylate group.

The salt of the organic compound A is generally dissolved in water or an aqueous medium comprising one or more hydrophilic solvents, and the product obtained on conclusion of step a) is then introduced into the solution of organic compound A.

The mixture is preferably left at room temperature until the ion exchange has taken place.

On conclusion of the reaction, the mixture is typically filtered and the recovered solid is washed with an organic solvent such as ethanol, and then dried to give a fluorescent particulate material.

The preparation process of the invention is illustrated in a nonlimiting manner by means of the examples that follow.

Applications

According to another aspect, the present invention relates to a cosmetic composition comprising, in a physiologically acceptable medium, at least one material in accordance with the invention.

The term “physiologically acceptable medium” is intended to denote a medium that is particularly suitable for the application of a cosmetic composition of the invention to the skin or the lips. The physiologically acceptable medium is generally adapted to the nature of the support onto which the composition has to be applied, and also to the appearance under which the composition has to be packaged.

The cosmetic composition according to the invention preferably comprises a content of material in accordance with the invention of from 0.0001% to 90%, preferably from 0.001% to 50%, preferentially from 0.01% to 25% and advantageously from 0.1% to 10%, by weight relative to the weight of said composition.

The composition may also comprise cosmetic additives such as fragrances, preserving agents, film-forming polymers, UV-screening agents, thickeners, water, oils, waxes, organic solvents, dyestuffs and fillers.

According to another aspect, the present invention relates to the use of the material in accordance with the invention, as an agent for lightening keratin materials and/or for reducing the appearance of the imperfections and/or relief irregularities of keratin materials.

The term “keratin materials” in particular means the skin, especially of the body, the hands, the neck, the face and the lips.

According to another aspect, the present invention relates to a non-therapeutic cosmetic process for treating keratin materials, comprising a step of applying a cosmetic composition in accordance with the invention to said keratin materials.

The non-therapeutic cosmetic process of the invention is advantageously for lightening said keratin materials.

The non-therapeutic cosmetic process of the invention is advantageously for reducing the appearance of skin imperfections, such as hyperpigmented or bleached marks, and/or skin relief irregularities, such as dilated pores and wrinkles.

As emerges from the examples below, it was in fact observed that the material in accordance with the invention is particularly suitable for lightening the skin immediately, while at the same time maintaining a very natural and uniform appearance, without any covering marks.

It was also observed that the material in accordance with the invention is particularly suitable for effectively and perceptibly masking skin relief irregularities immediately, especially as regards wrinkles, while at the same time maintaining a very natural skin appearance, without any covering marks.

It was also observed that the material in accordance with the invention is particularly suitable for giving the skin color homogeneity, by masking its colorimetric imperfections due to dyschromia marks or hypervascularized blood capillaries, while at the same time maintaining a very natural appearance of the skin, without any covering marks.

### EXAMPLES

Starting Materials
Silica H33 (Sunsphere H33 from AGC SI-TECH) having a porosity of 22.6 nm,
Silica H53 (Sunsphere H53 from AGC SI-TECH) having a porosity of 22.4 nm,
Silica H51 (Sunsphere H51 from AGC SI-TECH) having a porosity of 4.5 nm,
3-(Trimethoxysilyl)-1-chloropropyl,
N-Methylimidazole,
N-Trimethoxysilylpropyl-N,N,N-trimethylammonium chloride (as a solution at 50% by weight in methanol, Gelest),
N-Trimethoxysilylundecyl-N,N,N-tributylammonium bromide (as a solution at 25% by weight in dimethylformamide, Gelest),
4-Trimethoxysilylethyl)benzyl-N,N,N-trimethylammonium chloride (as a solution at 60% by weight in methanol, Gelest),
N-Octadecyl-N,N-dimethyl(3-trimethoxysilylpropyl)ammonium chloride (as a solution at 60% by weight in methanol, Gelest),
N-Trimethoxysilylpropyl-N,N,N-tributylammonium chloride (as a solution at 50% by weight in methanol, Gelest),
N-Trimethoxysilylpropyl-N,N,N-tributylammonium bromide (as a solution at 50% by weight in methanol, Gelest),
11-bromo-undecyltrimethoxysilane (Gelest),
Tinopal® CBS-X (disodium 2-[(Z)-2-[4-[4-[(Z)-2-(2-sulfonatophenyl)ethenyl]phenyl] phenyl]ethenyl]benzenesulfonate, BASF).

### Example 1

### Synthesis of the Material P1

Spherical silica particles H33 (Sunsphere), pregrafted with a ligand L1 (3-methyl-1-[3-(trimethoxysilyl)propyl]-1H-imidazol-3-ium chloride), were placed in contact with Tinopal® CBS-X (BASF) to form the material P1 in accordance with the invention.

The overall synthetic scheme is represented below:

### Step 1: Synthesis of the Ligand L

40.56 g of 3-(trimethoxysilyl)-1l-chloropropane (0.2 mol, 1 eq.) and 24.89 g of N-methylimidazole (0.3 mol, 1.5 eq.) were placed in a 250 mL three-necked flask under argon.

The mixture was stirred using a magnetic bar under a stream of argon for 1 hour at room temperature. The mixture was then heated (IKA hotplate+DrySyn aluminum heating block), under a gentle stream of argon, for 24 hours at 78° C. The mixture was then allowed to cool to room temperature.

The ligand L1 was obtained in a purity of 88.8% (measured by NMR), the remaining 11.2% corresponding to the starting N-methylimidazole.

### Step 2: Grafting of the Ligand L1 onto the Silica H33

3.17 g of the crude product (at 88.8% of L1) obtained on conclusion of step 1 (i.e. 2.81 g of pure ligand L1) were placed in a 250 mL three-necked flask, followed by addition of silica H33 (5.83 g, i.e. 0.51 g of ligand L1 per g of silica) and heptane (140 mL).

The mixture was stirred using a magnetic bar, under argon, and heated (IKA hotplate+DrySyn aluminum heating block) at 90° C. for 21 hours. The mixture was then allowed to cool to room temperature. 140 mL of heptane were added and the mixture was filtered through a Bichner funnel and glass microfiber of porosity 0.3 μm and diameter 70 mm. The solid filtered off was washed with 140 mL of ethanol and dried under vacuum at 40° C. in an oven (theoretical mass=7.9 g, mass obtained=7.5 g, yield=95%, grafting yield=96.6%).

### Step 3: Cl⁻/Tinopal® CBS-X Ion Exchange

2.02 g of Tinopal® CBS-X and 83 ml of demineralized water were placed in a 250 mL glass flask. The mixture was stirred using a magnetic bar for 1 hour at room temperature to the point of complete dissolution of the Tinopal® CBS-X. 5 g of silica grafted with the ligand L1 obtained on conclusion of step 2 were added and the mixture was stirred for 3 days at room temperature, and then filtered through a Bichner funnel and glass microfiber of porosity 0.3 μm and diameter 70 mm.

The solid filtered off was washed with 100 mL of water and then dried under vacuum at 40° C. in an oven to give the material P1 in the form of particles (5.8 g, exchange yield=80.6%).

### Example 2

### Synthesis of the Material P2

Spherical silica particles H53, pregrafted with the ligand L1 (3-methyl-1-[3-(trimethoxysilyl)propyl]-1H-imidazol-3-ium chloride), were placed in contact with Tinopal® CBS-X to form the material P2 in accordance with the invention according to a process equivalent to that described in Example 1, replacing the silica H33 with the silica H53.

### Step 1: Grafting of the Ligand L1 onto the Silica H53

2 L of heptane were placed in a jacketed 6 L reactor equipped with a reflux/distillation head and a condenser. The reactor was stirred at 100 rpm. 91.6 g of the crude product (at 87.4% of L1) obtained according to a process corresponding to step 1 of Example 1 (i.e. 80.28 g of pure ligand L1) were introduced, followed by addition of silica H53 (166.6 g, i.e. 0.51 g of ligand L1 per g of silica). Finally, 2 L of heptane were added.

The mixture was stirred under argon and heated in distillation mode (from 85° C. to 92° C.) for 7 hours. The mixture was then allowed to cool to room temperature. The product was extracted from the reactor and the reactor was washed with 4 L of ethanol. The whole (8 L) was filtered through a Bichner funnel and glass microfiber of porosity 0.3 μm. The solid filtered off was taken up in 4 L of ethanol and then filtered again and dried under vacuum at 40° C. in an oven (synthetic yield=96%, grafting yield (measured by TGA)=100%).

### Step 2: Cl⁻/Tinopal® CBS-X Ion Exchange

14.1 g of Tinopal CBS-X and 580 ml of demineralized water were placed in a 1 L reactor. The mixture was stirred for 1 hour at room temperature to the point of complete dissolution of the Tinopal® CBS-X. 35 g of silica grafted with the ligand L1 obtained on conclusion of step 1 were added and the mixture was stirred for 3 days at room temperature, and then filtered through a Bichner funnel and glass microfiber of porosity 0.3 μm.

The solid filtered off was washed with 500 mL of water and then 500 ml of ethanol, then dried under vacuum at 40° C. in an oven to give the material P2 in the form of particles (39.8 g).

### Example 3

### Synthesis of the Material P3

Spherical silica particles H51, pregrafted with the ligand L1 (3-methyl-1-[3-(trimethoxysilyl)propyl]-1H-imidazol-3-ium chloride), were placed in contact with Tinopal® CBS-X to form the material P3 in accordance with the invention according to a process equivalent to that described in Example 1, replacing the silica H33 with the silica H51.

### Example 4

### Synthesis of the Material P4

Spherical silica particles H33 (Sunsphere H33), pregrafted with the ligand L2 (1-(trimethoxysilyl)propyl-3-N,N,N-trimethylammonium chloride), were placed in contact with Tinopal® CBS-X (BASF) to form the material P4 in accordance with the invention, according to a process equivalent to that described in Example 1, replacing the ligand L1 with the ligand L2.

The overall synthetic scheme is represented below:

### Step 1: Grafting of the Ligand L2 onto the Silica H33

9.15 g of ligand L2 were placed in a 250 mL three-necked flask, followed by addition of silica H33 (17.49 g) and heptane (420 mL).

The mixture was stirred using a magnetic bar, under argon, and heated (IKA hotplate+DrySyn aluminum heating block) at 90° C. for 21 hours. The mixture was then allowed to cool to room temperature. 140 mL of heptane were added and the mixture was filtered through a Büchner funnel and glass microfiber of porosity 0.3 μm and diameter 70 mm. The solid filtered off was washed with 140 mL of ethanol and dried under vacuum at 40° C. in an oven (theoretical mass=22.41 g, mass obtained=19.7 g, yield=88%, grafting yield=91.7%).

### Step 2: Cl⁻/Tinopal CBS-X Ion Exchange

7.3 g of Tinopal CBS-X and 300 ml of demineralized water were placed in a 250 mL glass flask. The mixture was stirred using a magnetic bar for 1 hour at room temperature to the point of complete dissolution of the Tinopal CBS-X.

18 g of silica grafted with the ligand L2 obtained on conclusion of step 2 were added and the mixture was stirred for 3 days at room temperature, and then filtered through a Büchner funnel and glass microfiber of porosity 0.3 μm and diameter 70 mm.

The solid filtered off was washed with 100 mL of water and then dried under vacuum at 40° C. in an oven to give the material P4 in the form of particles (20 g, exchange yield=81.6%).

### Example 5

### Synthesis of the Material P5

Spherical silica particles H53, pregrafted with the ligand L2, were placed in contact with Tinopal® CBS-X to form the material P5 in accordance with the invention, according to a process equivalent to that described in Example 4, replacing the silica H33 with the silica H53.

### Example 6

### Synthesis of the Material P6

Spherical silica particles H51, pregrafted with the ligand L2, were placed in contact with Tinopal® CBS-X to form the material P6 in accordance with the invention, according to a process equivalent to that described in Example 4, replacing the silica H33 with the silica H51.

### Example 7

### Synthesis of the Material P7

Spherical silica particles H53, pregrafted with the ligand L3 (N-Trimethoxysilylundecyl-N,N,N-tributylammonium bromide), were placed in contact with Tinopal® CBS-X to form the material P7 in accordance with the invention, according to a process equivalent to that described in Example 2, replacing the ligand L1 with the ligand L3.

### Example 8

### Synthesis of the Material P8

Spherical silica particles H53, pregrafted with the ligand L4 (4-(trimethoxysilylethyl)benzyltrimethylammonium chloride), were placed in contact with Tinopal® CBS-X to form the material P8 in accordance with the invention, according to a process equivalent to that described in Example 2, replacing the ligand L1 with the ligand L4.

### Example 9

### Synthesis of the Material P9

Spherical silica particles H53, pregrafted with the ligand L5 (N,N-dimethyl-N(3-(trimethoxysilyl)propyl)octadecane-1-ammonium chloride), were placed in contact with Tinopal® CBS-X to form the material P9 in accordance with the invention, according to a process equivalent to that described in Example 2, replacing the ligand L1 with the ligand L5.

### Example 10

### Synthesis of the Material P10

Spherical silica particles H53, pregrafted with the ligand L6 (N-Trimethoxysilylpropyl-N,N,N-tributylammonium chloride), were placed in contact with Tinopal® CBS-X to form the material P10 in accordance with the invention, according to a process equivalent to that described in Example 2, replacing the ligand L1 with the ligand L6.

### Example 11

### Synthesis of the Material P11

Spherical silica particles H53, pregrafted with the ligand L7 (11-dimethylimidazolium undecyltrimethoxysilane bromide), were placed in contact with Tinopal® CBS-X to form the material P11 in accordance with the invention, according to a process equivalent to that described in Example 2, replacing the ligand L1 with the ligand L7.

### Synthesis of the Ligand L7

71.01 g of 11-bromoundecyltrimethoxysilane (0.2 mol, 1 eq.) and 24.89 g of N-methylimidazole (0.3 mol, 1.5 eq.) were placed in a 250 mL three-necked flask under argon.

The mixture was stirred using a magnetic bar under a stream of argon for 1 hour at room temperature. The mixture was then heated (IKA hotplate+DrySyn aluminum heating block), under a gentle stream of argon, for 24 hours at 78° C. The mixture was then allowed to cool to room temperature.

### Example 12

### Evaluation of the Haze, Transparency and Lightness Power of Materials P1 to P11

Eleven compositions were prepared comprising 3% by weight of each of the materials P1 to P11 of Examples 1 to 11 by mixing the material with the base composition B1 below (3% of the material and 97% of the base B1):

Base B1 (Weight %):

| | |
|---|---|
| Cetyl alcohol | 1.25% |
| Triethanolamine | 0.375% |
| Polyethylene stearate containing 40 ethylene oxide units | 3.125% |
| 1,2-Octanediol | 0.625% |
| Hydrogenated polyisobutene | 6.25% |
| (Parleam from NOF Corporation) | |
| Isohexadecane | 18.75% |
| Mixture of glyceryl stearate and PEG-100 stearate | 3.125% |
| (Arlacel ® 165 FL from Croda) | |
| Carboxyvinyl polymer | 0.375% |
| (Carbopol 981 polymer from Lubrizol) | |
| Stearyl alcohol | 1.25% |
| Preserving agent | qs |
| Water | qs 100% |

Similar compositions were also prepared with the untreated silica Sunsphere H33 and Sunsphere H51.

Each composition was then applied onto a polyester film sold under the reference 100045425 by the company Byk, using a film spreader to deposit a coat 50 μm thick, which was left to dry for 30 minutes at room temperature (25° C.). The transparency, haze and lightness measurements were taken on the dry deposit obtained, using a Hazegard Plus machine from Bye Additive & Instruments.

The following results were obtained:

| Composition | Transparency | Haze | Lightness |
|---|---|---|---|
| B1 + P1 | 93.14 | 86 | 14.64 |
| B1 + P2 | 92.72 | 83.66 | 11.28 |
| B1 + P3 | 91.88 | 62.26 | 23.3 |
| B1 + P4 | 92.76 | 91.76 | 16.28 |
| B1 + P5 | 91.52 | 83.22 | 8.6 |
| B1 + P6 | 92.02 | 72.04 | 21.62 |
| B1 + P7 | 92.3 | 87.4 | — |
| B1 + P8 | 91.6 | 61.9 | — |
| B1 + P9 | 92.3 | 87.4 | — |
| B1 + P10 | 91.4 | 71.1 | — |
| B1 + P11 | 91.9 | 80.1 | — |
| Silica H33 | 91.02 | 57.56 | 17.28 |
| Silica H51 | 91.06 | 55.72 | 16.32 |

The results obtained show that the deposits obtained have good natural transparency (between 91 and 94).

Furthermore, the deposits obtained have a high haze value (greater than 60), which shows that the materials in accordance with the invention have a good soft-focus effect and thus good masking of imperfections. The haze value of the materials according to the invention is greater than that of the untreated silica, which shows that these materials according to the invention have a better soft-focus effect and better masking of imperfections.

The deposits also have low lightness (less than 24), thus contributing toward a natural appearance of the deposit.

### Example 13

### Evaluation of the Lightening Effect of Materials P1, P4 and P5 to P11

Eight compositions were prepared comprising each of the materials P1, P4 and P5 to P11 by mixing the material with the base composition B2 below.

Base B2 (Weight %):

| | |
|---|---|
| Acetylated ethylene glycol stearate | 0.51% |
| Cyclopentadimethylsiloxane | 10.35% |
| Propylene glycol | 5% |
| Smectite in cyclopentadimethylsiloxane and ethanol | 5.82% |
| (18/77/5) (BENTONE GEL VS 5 V from Elementis) | |
| Magnesium sulphate | 0.7% |
| Mixture of Oxyethylenated Poly | 6.55% |
| methylcetyldimethylsiloxane, Poly glycerol | |
| isostearate and Hexyl laurate | |
| (Abil WE 09 from Evonik Goldschmidt) | |
| Polydimethylsiloxane (10 cSt) | 2.91% |
| N-oleyl dihydrosphingosine | 0.03% |
| Isododecane | 1.89% |
| Preserving agent | qs |
| Water | qsp 100% |

Compositions are prepared with such a material content that the amount of fluorescent Tinopal® CBS-X molecules is equal to 1.5 mmole, corresponding for example to weight contents of 3.22% for material P1 and 2.59% for material P4.

Two compositions comprising respectively 1.5% by weight of a mixture of Sunsphere H33+Tinopal® CBS-X and 1.5% by weight of a mixture of Sunsphere H53+Tinopal® CBS-X were also prepared.

Each composition, and also the base B2 alone, was then applied onto a glossy multizone cosmetic chart sold under the reference 25C by the company Leneta, using a film spreader to deposit a coat 50 μm thick, which was left to dry for 30 minutes at room temperature (25° C.). The multizone chart Leneta comprises 7 zones of different colours going from white (zone 1) to black (zone 7) going through different colours corresponding to different skin complexions (zones 2 to 6).

L a b colorimetry measurements were taken on each zone of the dry deposit obtained, using a Nikon D7000 camera, aperture 6.3, speed ⅛, with a D65 angular light.

For each colorimetric measurement, the difference A between the value obtained by applicating of the material and the value obtained by application of the base B2. The lightening properties of the applied materials is thus evaluated.

The following results were obtained for zones 2, 3 and 4, which are the most relevant.

| | Material | | | | | | | |
|---|---|---|---|---|---|---|---|---|
| | content | | | | | | | |
| Composition | (wt %) | Zone | L* | a* | b* | ΔL | Δa | Δb |
| Base B2 | — | 1 | 95.12 | −1.71 | 4.67 | 0.26 | −0.15 | 0.05 |
| Base B2 | — | 7 | 7.87 | −4.52 | 3.46 | −3.14 | −4.54 | 0.71 |
| Base B2 | — | 2 | 79.00 | 10.13 | 15.73 | −0.28 | 0.36 | 0.29 |
| Base B2 | — | 3 | 66.37 | 15.03 | 21.64 | −0.57 | 1.37 | 0.31 |
| Base B2 | — | 4 | 71.36 | 18.87 | 31.53 | −0.19 | 0.09 | 1.83 |
| P1 (ex 1; | 3.22 | 1 | 95.97 | −0.47 | −1.47 | 0.85 | 1.24 | −6.14 |
| H33-L1- | | | | | | | | |
| TCBSX) | | | | | | | | |
| P1 (ex 1) | 3.22 | 7 | 21.50 | 3.73 | −9.55 | 13.64 | 8.25 | −13.01 |
| P1 (ex 1) | 3.22 | 2 | 79.87 | 8.45 | 10.52 | 0.87 | −1.68 | −5.21 |
| P1 (ex 1) | 3.22 | 3 | 67.72 | 13.82 | 13.81 | 1.35 | −1.21 | −7.84 |
| P1 (ex 1) | 3.22 | 4 | 72.84 | 18.17 | 24.93 | 1.48 | −0.69 | −6.61 |
| P4 (ex 4; | 2.59 | 1 | 96.57 | −0.58 | −1.14 | 1.45 | 1.13 | −5.81 |
| H33-L2- | | | | | | | | |
| TCBSX) | | | | | | | | |
| P4 (ex 4) | 2.59 | 7 | 20.63 | 3.74 | −8.44 | 12.77 | 8.26 | −11.90 |
| P4 (ex 4) | 2.59 | 2 | 80.48 | 8.75 | 11.74 | 1.48 | −1.38 | −3.99 |
| P4 (ex 4) | 2.59 | 3 | 68.20 | 16.12 | 15.38 | 1.83 | 1.09 | −6.26 |
| P4 (ex 4) | 2.59 | 4 | 73.09 | 19.86 | 25.72 | 1.73 | 1.00 | −5.82 |
| Sunsphere | 1.5 | 1 | 96.67 | −1.60 | 2.26 | 1.55 | 0.11 | −2.41 |
| H33 + | | | | | | | | |
| TCBS-X | | | | | | | | |
| Sunsphere | 1.5 | 7 | 22.79 | 3.03 | −8.13 | 14.92 | 7.55 | −11.59 |
| H33 + | | | | | | | | |
| TCBS-X | | | | | | | | |
| Sunsphere | 1.5 | 2 | 80.74 | 9.41 | 14.26 | 1.74 | −0.72 | −1.47 |
| H33 + | | | | | | | | |
| TCBS-X | | | | | | | | |
| Sunsphere | 1.5 | 3 | 68.43 | 14.62 | 16.93 | 2.05 | −0.41 | −4.72 |
| H33 + | | | | | | | | |
| TCBS-X | | | | | | | | |
| Sunsphere | 1.5 | 4 | 73.57 | 19.28 | 27.33 | 2.21 | 0.42 | −4.20 |
| H33 + | | | | | | | | |
| TCBS-X | | | | | | | | |
| P5 (ex 5; | | 1 | 95.95 | −0.32 | −1.55 | 0.85 | −0.05 | −4.54 |
| H53 L2 | | | | | | | | |
| TCBS-X) | | | | | | | | |
| P5 (ex 5) | | 7 | 22.56 | 0.58 | −5.12 | −3.28 | −0.81 | −5.48 |
| P5 (ex 5) | | 2 | 82.17 | 7.82 | 9.63 | 3.31 | −1.43 | −5.74 |
| P5 (ex 5) | | 3 | 68.95 | 13.19 | 14.36 | 3.59 | −0.07 | −6.51 |
| P5 (ex 5) | | 4 | 73.81 | 18.32 | 23.07 | 2.29 | −0.63 | −4.44 |
| Sunsphere | | 1 | 95.61 | −0.77 | 1.85 | 0.51 | −0.5 | −1.14 |
| H53 + | | | | | | | | |
| TCBS-X | | | | | | | | |
| Sunsphere | | 7 | 25.03 | 1.45 | −3.82 | −0.80 | 0.05 | −4.18 |
| H53 + | | | | | | | | |
| TCBS-X | | | | | | | | |
| Sunsphere | | 2 | 81.09 | 8.33 | 13.00 | 2.24 | −0.92 | −2.36 |
| H53 + | | | | | | | | |
| TCBS-X | | | | | | | | |
| Sunsphere | | 3 | 69.07 | 12.56 | 17.33 | 3.72 | −0.7 | −3.54 |
| H53 + | | | | | | | | |
| TCBS-X | | | | | | | | |
| Sunsphere | | 4 | 73.68 | 18.51 | 25.41 | 2.16 | −0.43 | −2.11 |
| H53 + | | | | | | | | |
| TCBS-X | | | | | | | | |
| P7 ( ex 7; | | 1 | 95.40 | 0.30 | −2.22 | 0.31 | 0.57 | −5.2 |
| H53 L3 | | | | | | | | |
| TCBS-X) | | | | | | | | |
| P7 (ex 7) | | 7 | 22.81 | 1.48 | −5.02 | −3.02 | 0.09 | −5.38 |
| P7 (ex 7) | | 2 | 81.63 | 8.27 | 9.52 | 2.77 | −0.98 | −5.84 |
| P7 (ex 7) | | 3 | 68.97 | 13.27 | 14.18 | 3.62 | 0.01 | −6.68 |
| P7 (ex 7) | | 4 | 73.51 | 18.21 | 22.75 | 1.99 | −0.74 | −4.76 |
| P8 (ex 8; | | 1 | 95.14 | −0.11 | −1.32 | −0.25 | 0.15 | −4.32 |
| H53 L4 | | | | | | | | |
| TCBS-X) | | | | | | | | |
| P8 (ex 8) | | 7 | 22.76 | 1.08 | −3.56 | 2.63 | −0.31 | −3.92 |
| P8 (ex 8) | | 2 | 81.5 | 7.96 | 10.42 | −0.63 | −1.29 | −4.94 |
| P8 (ex 8) | | 3 | 68.81 | 12.87 | 15.12 | 0.58 | −0.38 | −5.75 |
| P8 (ex 8) | | 4 | 73.50 | 18.15 | 23.5 | −0.17 | −0.80 | −4.02 |
| P9 (ex 9; | | 1 | 95.15 | 0.18 | −0.722 | 0.06 | 0.45 | −3.72 |
| H53 L5 | | | | | | | | |
| TCBS-X) | | | | | | | | |
| P9 (ex 9) | | 7 | 23.67 | 1.64 | −1.95 | −2.16 | 0.25 | −2.32 |
| P9 (ex 9) | | 2 | 81.53 | 8.46 | 11.16 | 2.68 | −0.8 | −4.20 |
| P9 (ex 9) | | 3 | 69.00 | 12.81 | 16.21 | 3.65 | −0.44 | −4.66 |
| P9 (ex 9) | | 4 | 73.61 | 18.36 | 24.25 | 2.09 | −0.59 | −3.26 |
| P10 (ex10; | | 1 | 99.28 | −0.03 | −0.53 | 0.19 | 0.24 | −3.52 |
| H53 L6 | | | | | | | | |
| TCBS-X) | | | | | | | | |
| P10 (ex10) | | 7 | 22.24 | 1.23 | −3.46 | −3.59 | −0.16 | −3.82 |
| P10 (ex10) | | 2 | 81.65 | 8.47 | 11.36 | 2.79 | −0.78 | −4.01 |
| P10 (ex10) | | 3 | 68.85 | 13.00 | 16.56 | 3.49 | −0.25 | −4.31 |
| P10 (ex10) | | 4 | 73.46 | 18.66 | 24.77 | 1.94 | −0.29 | −2.74 |
| P11 (ex11; | | 1 | 96.23 | 0.01 | −2.01 | 1.14 | 0.28 | −5.01 |
| H53 L7 | | | | | | | | |
| TCBS-X) | | | | | | | | |
| P11 (ex11) | | 7 | 23.09 | 1.15 | −4.98 | −2.75 | −0.23 | −5.35 |
| P11 (ex11) | | 2 | 82.31 | 7.94 | 9.61 | 3.45 | −1.31 | −5.75 |
| P11 (ex11) | | 3 | 69.83 | 13.09 | 14.25 | 4.48 | −0.16 | −6.62 |
| P11 (ex11) | | 4 | 74.43 | 18.28 | 23.06 | 2.91 | −0.67 | −4.46 |
| BN | | 1 | 94.23 | −0.65 | 2.84 | | | |
| BN | | 7 | 29.27 | 0.24 | −2.00 | | | |
| BN | | 2 | 80.01 | 8.72 | 15.73 | | | |
| BN | | 3 | 66.92 | 12.69 | 19.06 | | | |
| BN | | 4 | 72.03 | 19.17 | 28.07 | | | |

BN: Bore Nitride sold under the tradename BORON NITRIDE POWDER TRES BN PUHP 3002 from Saint-Gobain Ceramics.
TCBS-X: Tinapol ® CBS-X

The results obtained show that the decrease of Ab is greater for the materials according to the invention than for the mixtures Sunsphere H33+TCBS-X and Sunsphere H53+TCBS-X, which means a greater decrease of the yellow tint.

An increase of ΔL means an increase of the lightness of the material and thus a lightening of the tint after the composition containing said material is applied.

Thus, these measurements show that the application of the materials according to the invention confer a lightening effect to the skin when applied, while having a natural appearance.

It has further been noticed that materials P1 and P6 both prepared with Sunsphere H51 (having a porosity of 4.5 nm) do not confer a lightening effect to the skin.

1: Fluorescent particulate material, formed from particles of an oxide of an element M, some of the atoms M of which bear a cationic group of formula (I): 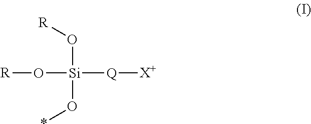
in which:
“*-” representing a covalent bond with an atom M,
M being chosen from titanium, iron, copper, zinc, zirconium, strontium, silicon, bismuth, cerium and mixtures thereof,
Q represents a linear or branched divalent C₁-C₁₂ alkylene radical, optionally interrupted with one or more oxygen atoms, and optionally substituted, or an optionally substituted C₆-C₁₀ arylene radical, or a divalent radical —R′—C₆H₄—R″, R′ and R″, which may be identical or different, represent a divalent C₁-C₄ alkylene radical and —C₆H₄— represents an aromatic divalent radical,
X⁺ represents a cationic organic group, and
the groups R, which may be identical or different, represent a hydrogen atom, a C₁-C₆ alkyl radical or a covalent bond with an atom M,
all or some of said cationic radicals of formula (I) being combined ionically with an anionic form of an organic compound A, which is fluorescent in the visible region, said anionic form of an organic compound A bearing at least one sulfonate group and/or at least one carboxylate group.

2: Material according to claim 1, in which the oxide of the element M is chosen from the group formed by TiO₂, Fe₂O₃, CuO, ZnO, ZrO₂, SiO₂, Bi₂O₃, CeO₂, SrO₂ and SiₓO_{y}, x and y being independently comprised between 0.1 and 2.

3: Material according to claim 1, in which the oxide of the element M is a silicon oxide.

4: Material according to claim 1, in which the particles are porous.

5: Material according to claim 1, in which the particles have a porosity of less than 1000 m²/g.

6: Material according to claim 1, in which the group X⁺ has the formula —N⁺R₁R₂R₃ in which groups R₁, R₂ and R₃, which may be identical or different, independently represent a linear or branched C₁-C₆ alkyl radical, optionally interrupted with one or more oxygen atoms, and optionally substituted, or an optionally substituted C₆-C₁₀ aryl group, one of the groups R₁, R₂ and R₃ eventually forming a linear or branched C₁-C₂₀ alkyl radical,
two of the groups R₁, R₂ and R₃ possibly forming together, with the nitrogen atom to which they are attached, an optionally substituted, 5- or 6-membered, saturated or unsaturated heterocycle.

7: Material according to claim 6, in which the groups R₁, R₂ and R₃, which may be identical or different, independently represent a methyl or ethyl group.

8: Material according to claim 1, in which the group X⁺ represents an unsaturated, 5- or 6-membered heterocyclic group, comprising one or more heteroatoms including a nitrogen atom bearing a positive charge, said group being optionally substituted.

9: Material according to claim 8, in which the group X⁺ is chosen from the group consisting of imidazolium, oxazolium, thiazolium, pyridinium, pyrimidinium, pyrazinium, benzimidazolium, benzoxazolium and benzothiazolium groups.

10: Material according to claim 1, in which Q represents a linear or branched divalent C₁-C₆ alkylene radical.

11: Material according to claim 1, in which the anionic form of the organic compound A bears from 2 to 4 groups chosen from the group consisting of the sulfonate group (—SO₃⁻), the carboxylate group (—COO⁻), and mixtures thereof.

12: Material according to claim 1, in which the organic compound A belongs to a chemical family chosen from the group consisting of xanthenes, coumarins, phenoxazines phenothiazines, naphthalimides, naphtholactams, lactamimides, quinacridones, epindolines, thio-epindolines, phthalimides, oxazolones, benzotriazoles, diphenylmaleimides, dibenzofurans, pyrimidines, triazines, 1,3,5-triazin-2-yl derivatives, pyrazines, triazoles, methines, distyrylbenzenes, distyrylbiphenyls, divinylstilbenes, triazinylaminostilbenes, stilbenyl-2H-triazoles, benzoxazoles, benzofurans, benzimidazoles, 1,3-diphenyl-2-pyrazolines, diketopyrrolopyrroles, perylenes and perylenemonoimides.

13: Material according to claim 1, in which the organic compound A belongs to a chemical family chosen from the group consisting of stilbenes, xanthenes, coumarins and naphthalimides.

14: Material according to claim 1, in which the organic compound A is a stilbene.

15: Material according to claim 1, in which the organic compound A corresponds to one of the following formulae: 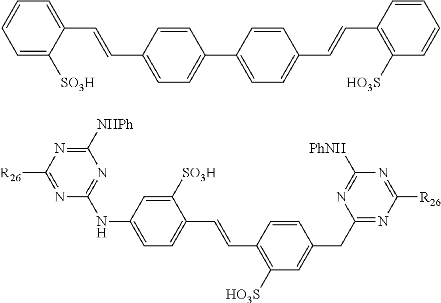
in which R₂₆ is a group chosen from OMe, NHMe, NHEt, NH—CH₂—CH₂OH, NH(CH₃)(CH₂—CH₂—OH), N-morpholinyl and NHPh.

16: Material according to claim 1, in which the particles are of spherical shape.

17: Material according to claim 1, in which the particles have a mean size ranging from 300 nm to 100 μm.

18: Material according to claim 1, in which the particles are porous silica particles having a mean pores size greater than 10 nm.

19: Process for preparing a material according to claim 1, comprising at least the steps consisting in:
a) placing particles of an oxide of an element M
in contact with at least one salt of formula (II): 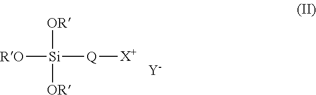
in which:
M, Q and X⁺ are as defined in claim 1,
R′ represents a linear or branched C₁-C₆ alkyl group, and
Y⁻ represents a monovalent anion,
under reaction conditions that are suitable for establishing covalent bonds between atoms M and cations of the salt of formula (II),
and
b) placing in contact the grafted particles formed in step a) with at least one salt of an organic compound A as defined in claim 1, under reaction conditions that are suitable for ionic exchange between anions Y⁻ and anionic forms of said organic compound A.

20: Process according to claim 19, in which the salt of the organic compound A is an alkali metal salt.

21: Cosmetic composition comprising, in a physiologically acceptable medium, at least one fluorescent particulate material according to claim 1.

22: Composition according to claim 21, in which the content of fluorescent particulate material is from 0.0001% to 90% by weight relative to the weight of said composition.

23: Method for reducing the appearance of imperfections and/or relief irregularities of keratin materials, comprising at least the step of using as an agent the material according to claim 1.

24: Method for lightening keratin materials, comprising at least the step of using as an agent the material according to claim 1.

25: Non-therapeutic cosmetic process for treating keratin materials, comprising a step of applying a cosmetic composition according to claim 21 to said keratin materials.

26: Process according to claim 25, for lightening said keratin materials, wherein the particles of the at least one particulate fluorescent material are porous silica articles having a mean pores size greater than 10 nm.

27: Process according to claim 25, for reducing the appearance of skin imperfections, and/or skin relief irregularities.
